(19) United States
(12) Patent Application Publication
(10) Pub. No.: US 2015/0065494 A1
(43) Pub. Date: Mar. 5, 2015

(54) AMINOSTYRYLBENZOFURAN DERIVATIVES AS INHIBITORS AGAINST BETA-AMYLOID FIBRIL FORMATION, AND PHARMACEUTICAL COMPOSITION COMPRISING SAME

(71) Applicant: KOREA INSTITUTE OF SCIENCE AND TECHNOLOGY, Seoul (KR)

(72) Inventors: Dong Jin Kim, Seoul (KR); Kyung Ho Yoo, Seoul (KR); Young Soo Kim, Gyeonggi-do (KR); Woong Seo Park, Seoul (KR); Yong Koo Kang, Seoul (KR); Hye Yun Kim, Seoul (KR); Yun Kyung Kim, Gangwon-do (KR); Ki Duk Park, Daejeon (KR); Maeng Sup Kim, Seoul (KR); Kwee Hyun Suh, Gyeonggi-do (KR); Young Gil Ahn, Gyeonggi-do (KR)

(21) Appl. No.: 14/389,609

(22) PCT Filed: Apr. 1, 2013
(86) PCT No.: PCT/KR2013/002699

§ 371 Date: Sep. 30, 2014

(30) Foreign Application Priority Data
Mar. 30, 2012 (KR) 10-2012-0033590

(51) Int. Cl. C07D 413/12; C07D 405/12; C07D 307/81

(52) U.S. Cl. CPC C07D 413/12; C07D 307/81; C07D 405/12
USPC 514/228.2; 548/215; 514/374; 549/469; 514/469; 544/153; 514/233.5; 548/525; 514/422; 546/284.1; 514/337; 544/376; 514/254.11; 544/62

(57) ABSTRACT

The present invention provides an aminostyrylbenzofuran compound of Formula (I), and a pharmaceutical composition comprising same. The pharmaceutical composition of the present invention comprising the compound of Formula (I), a pharmaceutically acceptable salt, an isomer, a hydrate, and a solvate thereof as an active ingredient can be useful for the prevention and treatment of degenerative brain diseases caused by accumulation of beta-amyloid.

### FIELD OF THE INVENTION

The present invention relates to a compound having an inhibitory activity against senile plaque formation induced by beta-amyloid or a pharmaceutically acceptable salt thereof, and a pharmaceutical composition comprising same for the prevention or treatment of degenerative brain diseases.

### BACKGROUND OF THE INVENTION

As the average life expectancy of the global population increases and population aging is taking place in many countries, the number of patients who are suffering from degenerative brain disease such as senile dementia (e.g., Alzheimer's disease, the most common form of senile dementia), stroke, and Parkinson's disease has increased rapidly. However, there is no method or drug available which directly targets the cause of such degenerative brain diseases, but only symptomatic drugs that alleviate the symptoms of the disease are available in the market.

Examples of commercially available medications for treating Alzheimer's dementia include TACKRIN™ (Warner-Lambert), ARICEPT™ (Eisai Co., Ltd.) and EXCELLON™ (Novartis), etc. The modes of action of these drugs, however, do not suppress the accumulation of beta-amyloid which is the cause of the disease. Instead, they increase the concentration of acetylcholine which is a neurotransmitter exists in synapse by inhibiting acetylcholine esterase, thereby improving cognitive function temporarily.

Alzheimer's disease is particularly dangerous form of senile dementia, and its pathogenesis is believed to be driven by beta-amyloid-induced neurotoxicity (Zlokovic, 2005; Mamikonyan et al., 2007). Specifically, a beta-amyloid precursor protein (APP) turns into a beta-amyloid 42 (Aβ42) monomer by β- and γ-secretase, and then the monomers aggregate to sequentially form oligomers, protofibril, fibril, and plaque, respectively. Thus, discovery of compounds that can specifically recognize and directly act on beta-amyloid, thereby preventing the fibril formation, could allow treating the fundamental cause of Alzheimer's disease.

As potential beta-amyloid, β- and γ-secretase inhibitors, metal chelators, beta-amyloid vaccines, statin-based drugs, nonsteroidal anti-inflammatory drugs have been studied. According to the study of beta-amyloid vaccine, in transgenic mice overexpressing beta-amyloid, a synthetic peptide called AN-1792 (Elan) was found to prevent the development of the senile plaque formation in young mice, while reducing the progression of the senile plaque formation in old mice (see Schenk, D. et al. Nature 1999, 400, 173). In other words, when transgenic mice with over-expressing beta-amyloid are administered with the beta-amyloid vaccine, it was observed that there have been generated antibodies capable of not only inhibiting beta-amyloid protein accumulation, but also removing amyloid plaques formed in the brain of the transgenic mice. This study shows that therapeutic agents which directly act on beta-amyloid to inhibit the formation of olygomers or senile plaques are useful for the prevention or treatment of senile dementia of the Alzheimer type.

Pharmaceutical drugs designed to deal with beta-amyloid are generally divided into two groups depending on the type of the target, mode of action and pharmacokinetics: therapeutic drugs and diagnostic molecular imaging agents.

Beta-amyloid fibril comprises 90% of beta-amyloid 40 (Aβ40) and 10% of beta-amyloid 42 (Aβ42) (see Bitan, G. et al., Proc. Natl. Sci. U.S.A 2003, 100, 330., and Jan, A. et al., J. Biol Chem. 2008, 283, 28176), and beta-amyloid 42 exhibits a strong neurotoxicity to induce apoptosis of brain cells. Therefore, beta-amyloid 42 is the major target of a therapeutic drug, while beta-amyloid 40 is that of a diagnostic agent. In terms of the mode of action, a therapeutic drug acts on soluble monomers and lower oligomers having an α-helix structure to inhibit the generation of insoluble oligomers which are 5 times more neurotoxic than fibrils. On the other hand, a diagnostic agent having a β-plated sheet structure exhibits a high binding affinity to insoluble oligomers. In terms of the pharmacokinetics, a therapeutic drug for degenerative brain diseases has different biodynamics from that of a diagnostic agent. Biodynamically, a diagnostic agent is required to have high absorption capable of quickly penetrating into the brain blood barrier (BBB) so that the diagnosis of a patient can be performed within the half life of the radioisotope used therein. Fast clearance (CL) of the diagnostic agent remaining after the diagnosis is also required to quantify the exact amount of the diagnostic agent bonded to the target, as well as to minimize non-specific binding of the diagnostic agent (see Mathis, C. A. et al., Curr. Pharm Design 2004, 10, 1469). In case of a therapeutic agent for brain disease, however, a high absorption leading to the penetration of the brain blood barrier preferred, similarly to the diagnostic agent, whereas optimized clearance is required so as to maximize its effect because high area under the concentration versus time curve (AUC) is required to show long durability in vivo unlike fast clearance of the diagnostic agent. In addition, degenerative brain diseases are chronic disease, and thus require a long-term drug therapy in general. Hence, linear pharmacokinetics of therapeutic agents, which is based on good absorbability and solubility when administered orally, are of great importance.

There are a number of compounds or extracts that are useful for the inhibition of the beta-amyloid fibril formation, and examples thereof include: detergents such as hexadecyl-N-methyl piperidinium (HMPBr) and the like; anti-cancer antibiotic agents such as doxorubicin and the like; benzofuran derivatives such as SKF-74652 (see Howlett, D. R. et al., Biochem. J. 1999, 343, 419) and the like; human acetylcholine secretases (HuAchE) such as propidium (see Bartolini, M. et al., Biochem. Pharmacol. 2003, 65, 407) and the like; a Ginko biloba extract, LB-152 (see Lin, S. et al., Bioorg. Med. Chem. Lett. 2004, 14, 1173); a curry extract named curcumin (see Yang, F. J. Biol. Chem. 2005, 280, 5892); and nordihydro guaiaretic acid (NDGA) (see Ono, K. et al., Biochem. Biophys. Res. Commun. 2005, 330, 111).

Among currently developed compounds having an inhibitory activity against the formation of beta-amyloid fibril, however, the compounds of a psudo-peptide type suffer from the problems of low bioavailability and poor stability due to high molecular weights thereof, and the anti-cancer antibiotic agents cause adverse side effects when administered over a long period of time. Further, it has been reported that said compounds and extracts have difficulties in meeting the requirements that a therapeutic agent for brain disease must be able to effectively penetrate through the brain blood barrier.

Accordingly, the inventors of the present invention have designed a compound with inhibitory activity against beta-amyloid fibril formation, which can effectively penetrate through the brain blood barrier, using low molecule compounds that are relatively safe from causing adverse side effects, and thus discovered a styrylbenzofuran compound which can inhibit the formation of beta-amyloid fibrils, particularly against beta-amyloid 42 (Korean Patent Laid-open Publication No. 2009-0129377).

However, the styrylbenzofuran compound failed to give consistent results in animal studies due to solubility problems. Thus, the present inventors have endeavored to develop a novel compound which is free from the solubility problems, and have found that a novel hydrophilic compound of aminostyrylbenzofuran for the prevention or treatment of degenerative brain diseases, which exhibits a high inhibitory effect on beta-amyloid fibril, especially beta-amyloid 42, as well as significantly enhanced solubility allowing good results in animal studies with linear pharmacokinetics.

### DISCLOSURE OF THE INVENTION

### Problems to be Solved by the Invention

Accordingly, it is an object of the present invention to provide a compound as an inhibitor against beta-amyloid fibril formation or a pharmaceutically acceptable salt thereof.

It is another object of the present invention to provide an inhibitor against beta-amyloid fibril formation comprising the compound or a pharmaceutically acceptable salt thereof as an active ingredient.

It is another object of the present invention to provide a pharmaceutical composition comprising the compound or a pharmaceutically acceptable salt thereof as an active ingredient for the prevention or treatment of degenerative brain diseases.

### Means for Solving the Problem

In order to accomplish the above object, the present invention provides a compound selected from the group consisting of an aminostyrylbenzofuran compound of Formula (I) below, a pharmaceutically acceptable salt, an isomer, a hydrate, and a solvate thereof:

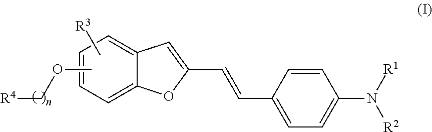

wherein

R¹ and R² are each independently hydrogen, C₁₋₆ alkyl, or C₃₋₈ cycloalkyl, wherein the C₁₋₆ alkyl or C₃₋₈ cycloalkyl is optionally substituted with one or more halogens;

R³ is hydrogen, C₁₋₃ alkyl or C₁₋₃ alkoxy;

R⁴ is hydrogen, NR⁵R⁶, C₃₋₈ heterocycloalkyl comprising 1 or more N, and randomly comprising O and S, or pyridyl, wherein the C₃₋₈ heterocycloalkyl or pyridyl is optionally substituted with one or more substituents selected from the group consisting of C₁₋₃ alkyl and oxo, and said R⁵ and R⁶ are each independently hydrogen or C₁₋₆ alkyl; and

n is an integer ranging from 1 to 4.

Further, the present invention provides an inhibitor against beta-amyloid fibril formation comprising the compound as an active ingredient.

Further, the present invention provides a pharmaceutical composition comprising the compound as an active ingredient for the prevention or treatment of degenerative brain diseases.

Further, the present invention provides a use of the compound as an active ingredient for the manufacture of a medicament for preventing or treating degenerative brain diseases.

Furthermore, the present invention provides a method for preventing or treating degenerative brain diseases, which comprises administering the compound as an active ingredient to a mammal in need thereof.

### Effect of the Invention

The aminostyrylbenzofuran-based compound according to the present invention shows a good inhibitory activity against beta-amyloid fibril formation, and is thus useful for the treatment of degenerative brain disease such as senile dementia, stroke, Parkinson's disease, etc.

### DETAILED DESCRIPTION OF THE INVENTION

Embodiments of the present invention are explained in detail hereinafter.

The term ‘alkyl’ as used herein refers to straight, cyclic, or branched hydrocarbon residues, unless otherwise indicated.

The term ‘cycloalkyl’ as used herein refers to cyclic alkyls including cyclopropyl, and others, unless otherwise indicated.

The term ‘heterocycloalkyl’ as used herein refers to cyclic alkyls including monocyclic, bicyclic or multicyclic alkyls, and others which contain one or more heteroatoms selected from O, N and S, unless otherwise indicated. Examples of monoheterocycloalkyl include piperidinyl, morpholinyl, thiamorpholinyl, pyrrolidinyl, oxazolidinyl, tetrahydrofuranyl, piperazinyl and similar groups thereof, but not limited thereto.

The compound in accordance with the present invention may also form a pharmaceutically acceptable salt. Such salt may be a nontoxic acid addition salt containing a pharmaceutically acceptable anion, but not limited thereto. For example, the salt may include acid addition salts formed by inorganic acids such as hydrochloric acid, sulfuric acid, nitric acid, phosphoric acid, hydrobromic acid, hydriodic acid, and the like; organic carbonic acids such as tartaric acid, formic acid, citric acid, acetic acid, trichloroacetic acid, trifluoroacetic acid, phenylacetic acid, gluconic acid, benzoic acid, hydroxybenzoic acid, glycolic acid, lactic acid, pyruvic acid, malonic acid, succinic acid, glutaric acid, fumaric acid, malic acid, mandelic acid, maleic acid, hydroxymaleic acid, ascorbic acid, palmitic acid, cinnamic acid, salicylic acid, and the like; and sulfonic acids such as methanesulfonic acid, benzenesulfonic acid, p-toluenesulfonic acid, naphthalenesulfonic acid, and the like. Among them, acid addition salts formed by sulfuric acid, methanesulfonic acid, hydrohalogenic acid, etc. are preferred.

Meanwhile, the compound according to the present invention can have an asymmetric carbon center, and thus may be present in the form of R or S isomer, racemic compounds, diastereomeric mixture, or individual diastereomer, such entire isomers and mixtures being included within the scope of the present invention.

In addition, solvates and hydrates of the compound of Formula (I) are included within the scope of the present invention.

In the aminostyrylbenzofuran compound of Formula (I) according to the present invention, preferably,

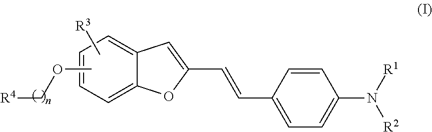

R¹ is methyl;

R² is methyl or 2-fluoro ethyl;

R³ is hydrogen or C₁₋₂ alkoxy;

R⁴ is hydrogen, dimethylamine, diethylamine, pyridin-2-yl, pyridin-3-yl, pyridin-4-yl, piperidin-1-yl, 1-methylpiperidin-2-yl, 1-methylpiperidin-3-yl, oxazolidin-3-yl, pyrrolidin-1-yl, 1-methylpyrrolidin-2-yl, morpholin-4-yl, (2S,6R)-2,6-dimethylmorpholin-4-yl, 1-methylpiperazin-4-yl, thiomorpholin-4-yl, or 1,1-dioxothiomorpholin-4-yl; and

n is an integer ranging from 1 to 3.

The examples of the preferred aminostyrylenzofuran compound or its derivatives according to the present invention are as follows. In addition to the derivatives, a pharmaceutically acceptable salt, an isomer, a hydrate, or a solvate thereof may be used.

1) (E)-6-(2-(3-oxazolidinyl)ethoxy)-2-(4-dimethylaminostyryl)benzofuran;

2) (E)-6-(2-dimethylaminoethoxy)-2-(4-dimethylaminostyryl)benzofuran;

3) (E)-6-(3-dimethylaminopropoxy)-2-(4-dimethylaminostyryl)benzofuran;

4) (E)-6-(3-diethylaminopropoxy)-2-(4-dimethylaminostyryl)benzofuran;

5) (E)-6-(3-(4-morpholinopropoxy))-2-(4-dimethylaminostyryl)benzofuran;

6) (E)-6-(2-(1-pyrrolidinyl)propoxy)-2-(4-dimethylaminostyryl)benzofuran;

7) (E)-6-(2-(1-methyl-2-pyrrolidinyl)ethoxy)-2-(4-dimethylaminostyryl)benzofuran;

8) (E)-6-(2-(4-morpholinoethoxy))-2-(4-dimethylaminostyryl)benzofuran;

9) (E)-6-(3-((2S,6R)-2,6-dimethylmorpholino)propoxy)-2-(4-dimethylaminostyryl)benzofuran;

10) (E)-6-(2-(1-piperidinyl)ethoxy)-2-(4-dimethylaminostyryl)benzofuran;

11) (E)-6-(3 -(1 -piperidinyl)propoxy)-2-(4-dimethylaminostyryl)benzofuran;

12) (E)-6-(1-methyl-2-piperidinylmethoxy)-2-(4-dimethylaminostyrypbenzofuran;

13) (E)-6-(1-methyl-3-piperidinylmethoxy)-2-(4-dimethylaminostyryl)benzofuran;

14) (E)-6-(3-(4-methyl-1-piperazinyl)propoxy)-2-(4-dimethylaminostyryl)benzofuran;

15) (E)-6-(3-(4-thiomorpholinopropoxy))-2-(4-dimethylaminostyryl)benzofuran;

16) (E)-6-(3-((4-thiomorpholine)-1,1-dioxide)propoxy)-2-(4-dimethylaminostyryl)benzofuran;

17) (E)-6-(3-(4-pyridinyl)propoxy)-2-(4-dimethylaminostyryl)benzofuran;

18) (E)-6-(3 -(3 -pyridinyl)propoxy)-2-(4-dimethylaminostyryl)benzofuran;

19) (E)-6-(3-(2-pyridinyl)propoxy)-2-(4-dimethylaminostyryl)benzofuran; and

20) (E)-6-(methoxy)-2-(4,4′-methyl(2-fluoroethyl)aminostyryl)benzofuran.

The compound of the present invention, which is selected from the group consisting of the compound of Formula (I), and a pharmaceutically acceptable salt, an isomer, a hydrate, and a solvate thereof, can inhibit beta-amyloid fibril formation and also effectively penetrate through the brain blood barrier, and is thus useful for the prevention and treatment of degenerative brain diseases caused by accumulation of beta-amyloid as an inhibitor against beta-amyloid fibril formation.

Accordingly, the present invention provides an inhibitor against beta-amyloid fibril formation comprising the compound selected from the group consisting of the compound of Formula (I), and a pharmaceutically acceptable salt, an isomer, a hydrate, and a solvate thereof, as an active ingredient.

In addition, the present invention provides a pharmaceutical composition for the prevention and treatment of degenerative brain diseases which comprises the compound of the present invention as an active ingredient.

Further, the present invention provides a use of the compound of the present invention as an active ingredient for the manufacture of a medicament for preventing or treating degenerative brain diseases.

Furthermore, the present invention provides a method for preventing or treating degenerative brain diseases, which comprises administering the compound of the present invention as an active ingredient to a mammal in need thereof.

Examples of the degenerative brain diseases which can be treated by the compound of the present invention include senile dementia (e.g., Alzheimer's disease), as well as stroke, Parkinson's disease, Huntington's disease, mad cow disease, and the like, which are deeply related with to accumulation of beta-amyloid fibril in the brain.

In the pharmaceutical composition of the present invention, the compound selected from the group consisting of the compound of Formula (I), and a pharmaceutically acceptable salt, an isomer, a hydrate, and a solvate thereof may be employed in an amount of 0.5 to 10% by weight, preferably 0.5 to 5% by weight, based on the total weight of the composition.

The pharmaceutical composition of the present invention may be sterilized and/or further comprise a preservative, a stabilizer, a wettable powder or an emulsifier, a supplement such as salt and/or buffer for osmotic pressure control, and other therapeutically acceptable additives. The pharmaceutical composition of the present invention may be formulated in accordance with conventional methods such as mixing, granulation and coating, and may be prepared in the form of various oral formulations, or parenteral formulations such as intramuscular, intravenous or subcutaneous administrations.

For oral formulations, the pharmaceutical composition of the present invention may be prepared in the form of tablets, pills, hard/soft capsules, liquid preparation, suspensions, emulsions, syrups, granules, and the like. The pharmaceutical composition of the present invention may further comprise a diluent (e.g., lactose, dextrose, sucrose, mannitol, sorbitol, cellulose and/or glycine), and a lubricating agent (e.g., silica, talc, stearic acid, magnesium stearate, calcium stearte and/or polyethylene glycol). The tablet may further comprise a binder (e.g., magnesium aluminum silicate, starch paste, gelatin, tragacanth, methylcellulose, carboxymethylcellulose sodium, polyvinylpyrrolidine, etc.); and it may further comprise a disintegrating agent (e.g., starch, agar, alginate or sodium salt thereof), a boiling mixture, an absorbent, a colorant, a flavoring agent and a sweetening agent, if necessary. For parenteral formulations, injectable formulation in the form of an isotonic aqueous solution and a suspension are preferred.

A proposed daily dose of the active ingredient of the inventive compound for administration to mammals, including humans, may be in the range of 0.01 to 30 mg/kg (body weight), preferably 0.1 to 10 mg/kg (body weight). The inventive compound may be administered in a single dose or in divided doses per day via oral or parenteral administration.

Hereinafter, an exemplary method for preparing the compound of the present invention is explained.

Following abbreviations are used in, preparation methods and Examples below:

THF: tetrahydrofuran
HCl: hydrochloric acid
EtOAc: ethylacetate
Boc-: tert-butoxycarbonyl
MeOH: methanol
TEA: triethylamine
TFA: trifluoroacetic acid
HBr: bromic acid
LiAlH₄: lithium aluminum hydride
DIPEA: N,N-diisopropylethylamine
DIAD: diisopropyl azodicarboxylate
AcCl: acetyl chloride
NaOH: sodium hydroxide
Hexane: n-hexane
DMSO: dimethyl sulfoxide
Dioxane: dioxane
MgSO₄: magnesium sulfate
MC: dichloromethane
NaHCO₃: sodium bicarbonate
Na₂SO₄: sodium sulfate

The compound of Formula (I) in accordance with the present invention, i.e., aminostyrylbenzofuran compound, may be prepared via Reaction Scheme 1 or other general reaction schemes for preparing aminostyrylbenzofuran compound:

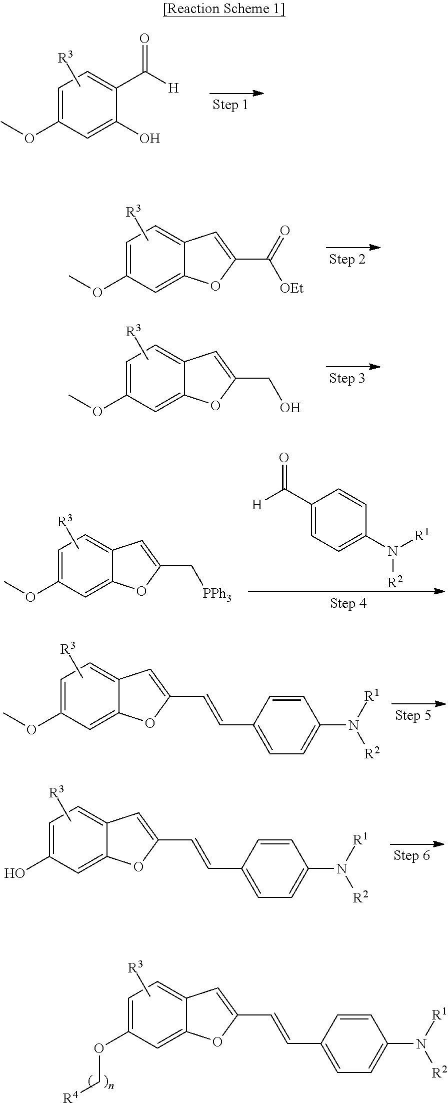

wherein R¹ to R⁴ and n are the same as defined in Formula (I).

In Reaction Scheme 1 above, additional steps may be conducted after Step 4 depending on the type of the residue of R¹ and R² groups. The above reaction processes are exemplified in following stepwise reaction.

In the stepwise processes, equivalent units are based on a standard equivalent unit, and solvents are expressed in mol per L of a standard equivalent unit.

<Step 1>

A starting material is prepared by adding aldehyde (1.0 equivalent, standard equivalent unit) to N,N-dimethylformamide (0.8 to 1.2 L/mol). Molecular sieves 4 Å (330 g/Kg) and potassium carbonate (2.0 to 2.5 equivalents) are added thereto, followed by stirring at 25 to 35° C. (the color of the solution changes from red to orange when potassium carbonate is added). Ethyl bromoacetate (1.8 to 2.2 equivalents) is slowly added to the resulting mixture, followed by stirring for 20 to 40 minutes at 25 to 35° C. The reaction solution is further stirred by refluxing the solution for 0.8 to 1.5 hrs at 160 to 190° C. (white solid is formed). The completion of the reaction is observed by thin layer chromatography, and then the resulting solution is added with potassium carbonate (2.0 to 2.5 equivalents), stirred at reflux for 1.5 to 2.5 hrs. The reaction solution is cooled down to 20 to 25° C. and filtered to remove impurities. The filtrate is extracted with EtOAc and water, and the organic layer is separated, dried over Na₂SO₄, and concentrated under reduced pressure. The resulting residue is solidified by diethyl ether, and filtered to obtain the title compound.

<Step 2>

The compound obtained in Step 1 (1.0 equivalent, standard equivalent unit) and sodium borohydride (3.0 to 3.5 equivalents) are dissolved in THF (3.0 to 3.5 L/mol), and stirred at reflux for 1.0 to 1.5 hrs. Me0H (0.8 to 1.2 L/mol) is slowly added over 2.5 to 3.5 hrs, and the resulting solution is stirred at reflux, followed by cooling to 20 to 25° C. Water (3.2 to 3.8 L/mol) is added thereto, and the resulting solution is stirred for 1 to 2 hrs at room temperature, followed by extraction with EtOAc. The organic layer is separated, dried over Na₂SO₄, and concentrated under reduced pressure to obtain the title compound.

<Step 3>

The compound obtained in Step 3 (1.0 equivalent, standard equivalent unit) and triphenylphosphine hydrobromide (1.0 to 1.1 equivalents) are added to acetonitrile (5.0 to 5.5 L/mol), and stirred at reflux for 1 to 2 hrs. The reaction solution is cooled to room temperature and concentrated under reduced pressure. The resulting residue is solidified by diethyl ether for 0.5 to 1.5 hrs, washed with diethyl ether, and filtered to obtain the title compound.

<Step 4>

The compound obtained in Step 3 (1.0 equivalent, standard equivalent unit) and potassium carbonate (1.8 to 2.4 equivalents) are dissolved in MeOH (5.0 to 5.4 L/mol), and stirred for 25 to 40 min at room temperature. Benzaldehyde substituted with R¹ and R² (1.0 to 1.2 equivalents) is dissolved in MeOH (0.70 to 0.78 L/mol) and the resulting solution is slowly added to the reaction solution, followed by stirring for 2.5 to 4.0 hrs in the dark conditions. The reaction solution is concentrated under reduced pressure in the dark, and extracted with EtOAc and water. The organic layer is separated, dried over Na₂SO₄, and concentrated under reduced pressure. The resulting residue is solidified by MeOH in the dark, washed with MeOH, and filtered to obtain the title compound.

<Step 5>

The compound obtained in Step 4 (1.0 equivalent, standard equivalent unit) and NaSEt (9.0 to 11.0 equivalents) are added to N,N-dimethylformamide (3.0 to 3.6 L/mol), and stirred at reflux for 1 to 2 hrs. The reaction solution is cooled to room temperature, added with an aqueous solution of sodium bicarbonate, and extracted with EtOAc. The organic layer is separated, washed with water and brine, dried over Na₂SO₄, and distilled under reduced pressure. The resulting residue is solidified by diethyl ether, washed with diethyl ether, and filtered to obtain the title compound.

<Step 6>

The compound obtained in Step 5 (1.0 equivalent, standard equivalent unit) and triphenylphosphine (2.0 to 2.2 equivalents) are stirred in THF (9.0 to 11 L/mol). Alcohol substituted with R⁴ (2.0 to 2.2 equivalents) and DIAD (2.0 to 2.2 equivalents) are added thereto, followed by stirring for 2.5 to 3.5 hrs at room temperature. The reaction mixture is extracted with EtOAc and water, and the organic layer is separated, and washed with brine. The organic layer is dried over Na₂SO₄, concentrated under reduced pressure. The resulting residue is solidified by EtOAc, washed with EtOAc, and filtered to obtain the title compound.

Hereinafter, the present invention is described more specifically by the following Examples, but these are provided only for illustration purposes, and the present invention is not limited thereto.

### EXAMPLE 1

### Preparation of (E)-6-(2-(3-oxazolidinyl)ethoxy)-2-(4-dimethylaminostyryl)benzofuran

<Step 1> Preparation of 6-methoxy-2-ethoxycarbonyl benzofuran

A solution was prepared by adding 2-hydroxy-4-methoxybenzaldehyde (1 kg, 6.57 mol) to N,N-dimethylformamide (7 L). Molecular sieves 4 Å (330 g) and potassium carbonate (2 kg, 14.47 mol) were added thereto, followed by stirring at room temperature (the color of the solution changed from red to orange when potassium carbonate was added). Ethyl bromoacetate (1.46 L, 13.14 mol) was slowly added to the resulting mixture, followed by further stirring for 30 minutes at room temperature. The reaction solution was further stirred by refluxing the solution for 1 hr at 175° C. (white solid was formed). The completion of the reaction was observed by thin layer chromatography, and then the resulting solution was added with potassium carbonate (2 kg, 14.47 mol), stirred at reflux for 2 hrs. The reaction solution was cooled down to room temperature, and filtered to remove impurities. The filtrate was extracted with EtOAc and water, and the organic layer was separated, dried over Na₂SO₄, and concentrated under reduced pressure. The resulting residue was solidified by diethyl ether, and filtered to obtain the title compound (684 g, 48%).

¹H NMR (DMSO-d6, 300 MHz) δ 7.66 (s, 1H), 7.64 (d, 111), 7.30 (d, 1H), 6.97 (dd, 1H), 4.32 (q, 2H), 3.82 (s, 3H), 1.31 (t, 3H)

<Step 2> Preparation of 6-methoxy-2-hydroxymethyl benzofuran

6-methoxy-2-ethoxycarbonyl benzofuran (309 g, 1.403 mol) obtained from Step 1 above and sodium borohydride (159.2 g, 4.209 mol) were dissolved in THF (6.2 L), and stirred at reflux for 1 hr. MeOH (1.2 L) was slowly added over 3 hrs, and the resulting solution was stirred at reflux, followed by cooling to room temperature. Water (4.8 L) was added thereto, and the resulting solution was stirred for 1 hr at room temperature, followed by extraction with EtOAc. The organic layer was separated, dried over Na₂SO₄, and concentrated under reduced pressure to obtain the title compound (250 g, 100%).

¹H NMR (CDCl₃, 300 MHz) δ 7.41-7.38 (d, 1H), 6.99 (s, 1H), 6.87-6.83 (dd, 1H), 6.57 (s, 1H), 4.72 (s, 2H), 3.84 (s, 3H)

<Step 3> Preparation of ((6-methoxybenzofuran-2-yl)methyl)triphenylphosphonium bromide

6-methoxy-2-hydroxymethyl benzofuran (250 g, 1.403 mol) obtained in Step 2 above and triphenylphosphine hydrobromide (491 g, 1.431 mol) were added to acetonitrile (7.5 L), and stirred at reflux for 1 hr. The reaction solution was cooled to room temperature and concentrated under reduced pressure. The resulting residue was solidified by diethyl ether for 1 hr, washed with the same solvent, and filtered to obtain the title compound (670 g, 96%).

¹H NMR (DMSO-d6, 300 MHz) δ 7.94-7.87 (m, 3H), 7.81-7.69 (m, 12H), 7.43-7.39 (d, 1H), 6.92 (s, 1H), 6.85-6.82 (dd, 1H), 6.62-6.61 (d, 1H), 5.63-5.58 (d, 2H), 3.75 (s, 3H)

<Step 4> Preparation of (E)-6-methoxy 2-(4-dimethylaminostyryl)benzofuran ((6-methoxybenzofuran-2-yl)methyl)triphenylphosphonium bromide (679 g, 1.35 mol) obtained in Step 3 above and potassium carbonate (373 g, 2.7 mol) were dissolved in MeOH (6.8 L), and stirred for 30 min at room temperature. A solution prepared by dissolving 4-(dimethylamino)benzaldehyde (201 g, 1.35 mol) in MeOH (1L) was slowly added thereto, and the resulting solution was further stirred for 3 hrs in the dark conditions. The reaction solution was concentrated under reduced pressure in the dark, and extracted with EtOAc and water. The organic layer was separated, dried over Na₂SO₄, and concentrated under reduced pressure. The resulting residue was solidified by MeOH in the dark, washed with MeOH, and filtered to obtain the title compound (144 g, 36%).

¹H NMR (DMSO-d6, 300 MHz) δ 7.46-7.42 (dd, 3H), 7.16-7.15 (d, 1H), 7.10-6.90 (q, 2H), 6.86-6.82 (dd, 1H), 6.74-6.71 (m, 2H), 3.81 (s, 3H), 2.94 (s, 6H)

<Step 5> Preparation of (E)-6-hydroxy 2-(4-dimethylaminostyryl)benzofuran

The (E)-6-methoxy 2-(4-dimethylaminostyryl)benzofuran (96 g, 0.327 mol) obtained in Step 4 above and NaSEt (275 g, 3.27 mol) were added N,N-dimethylformamide (1.1 L), and stirred at reflux for 1 hr. The reaction solution was cooled to room temperature, added with an aqueous solution of sodium bicarbonate, and extracted with EtOAc. The organic layer was separated, washed with water and brine, dried over Na₂SO₄, and distilled under reduced pressure. The resulting residue was solidified by diethyl ether, washed with diethyl ether, and filtered to obtain title compound (83 g, 91%).

¹H NMR (DMSO-d6, 300 MHz) δ 9.55 (brs, 1H), 7.44-7.41 (d, 2H), 7.33-7.31 (d, 1H), 7.06-6.87 (q, 2H), 6.87 (s, 1H), 6.73-6.65 (m, 4H), 2.94 (s, 6H)

MS (ESI⁺, m/z): 407 [M+H]⁺

<Step 6> Preparation of (E)-6-(2-(3-oxazolidinyl)ethoxy)-2-(4-dimethylaminostyryl)benzofuran

(E)-6-hydroxy 2-(4-dimethylaminostyryl)benzofuran (73 g, 0.262 mol) obtained in Step 5 above and triphenylphosphine (137 g, 0.523 mol) were stirred in THF (2.6 L). 2-(oxazolidyn-3-yl)ethanol (61.29 g, 0.523 mol) and DIAD (103 mL, 0.523 mol) were added thereto, followed by stirring for 3 hrs at room temperature. The reaction mixture was extracted with EtOAc and water, and the organic layer was separated, and washed with brine. The organic layer was dried over Na₂SO₄, concentrated under reduced pressure. The resulting residue was solidified by diethyl ether, washed with diethyl ether, and filtered to obtain the title compound (82 g, 77%).

¹H NMR (CDCl₃, 400 MHz) δ 7.41 (d, 2H), 7.34 (d, 1H), 7.17 (d, 1H), 7.02 (d, 1H), 6.84 (dd, 1H), 6.76 (d, 1H), 6.71 (d, 2H), 6.49 (s, 1H), 4.40 (s, 2H), 4.14 (t, 2H), 3.82 (t, 2H), 3.10 (t, 2H), 3.01 (t, 2H), 2.99 (s, 6H)

MS (ESI⁺, m/z): 379 [M+H]⁺

Hereinafter, the procedures of Example 1 were repeated by employing respective corresponding starting compounds to obtain the respective title compounds of Examples 2 to 19 having the following analytical data.

### EXAMPLE 2

### Preparation of (E)-6-(2-dimethylaminoethoxy)-2-(4-dimethylaminostyryl)benzofuran

¹H NMR (CDCl₃, 400 MHz) δ 7.41 (d, 2H), 7.34 (d, 1H), 7.16 (d, 1H), 7.02 (d, 1H), 6.85 (dd, 1H), 6.77 (d, 1H), 6.71 (d, 2H), 6.49 (s, 1H), 4.11 (t, 2H), 3.00 (s, 6H), 2.77 (t, 2H), 2.36 (s, 6H)

MS (ESI⁺, m/z): 351 [M+H]⁺

### EXAMPLE 3

### Preparation of (E)-6-(3-dimethylaminopropoxy)-2-(4-dimethylaminostyryl)benzofuran

¹H NMR (CDCl₃, 400 MHz) δ 7.41 (d, 2H), 7.34 (d, 1H), 7.16 (d, 1H), 7.00 (s, 1H), 6.81 (dd, 1H), 6.77 (d, 1H), 6.72 (d, 2H), 6.49 (s, 1H), 4.07 (t, 2H), 3.01 (s, 6H), 2.60 (br, 2H), 2.36 (s, 6H), 2.06 (m, 2H)

MS (ESI⁺, m/z): 365 [M+H]⁺

### EXAMPLE 4

### Preparation of (E)-6-(3-diethylaminopropoxy)-2-(4-dimethylaminostyryl)benzofuran

¹H NMR (CDCl₃, 400 MHz) δ 7.41 (d, 2H), 7.34 (d, 1H), 7.16 (d, 1H), 7.00 (s, 1H), 6.81 (dd, 1H), 6.77 (d, 1H), 6.71 (d, 2H), 6.48 (s, 1H), 4.06 (t, 2H), 3.00 (s, 6H), 2.72 (m, 6H), 2.05 (m, 2H), 1.12 (t, 6H)

MS (ESI⁺, m/z): 393 [M+H]⁺

### EXAMPLE 5

### Preparation of (E)-6-(3-(4-morpholinopropoxy))-2-(4-dimethylaminostyryl)benzofuran

¹H NMR (DMSO-d6, 300 MHz) δ 7.46-7.41 (t, 3H), 7.13 (s, 1H), 7.09-6.90 (q, 2H), 6.85-6.81 (dd, 1H), 6.74-6.65 (m, 3H), 4.08-4.03 (t, 2H), 3.59-3.56 (t, 4H), 2.94 (s, 6H), 2.46-2.41 (t, 2H), 2.37 (m, 4H), 1.94-1.87 (quin, 2H)

MS (ESI⁺, m/z): 407 [M+H]⁺

### EXAMPLE 6

### Preparation of (E)-6-(2-(1-pyrrolidinyl)propoxy)-2-(4-dimethylaminostyryl)benzofuran

MS (ESI⁺, m/z): 391 [M+H]⁺

### EXAMPLE 7

### Preparation of (E)-6-(2-(1-methyl-2-pyrrolidinypethoxy)-2-(4-dimethylaminostyrypbenzofuran

¹H NMR (CDCl₃, 400 MHz) δ 7.41 (d, 2H), 7.34 (d, 1H), 7.16 (d, 1H), 6.85 (d, 1H), 6.81 (dd, 1H), 6.77 (d, 1H), 6.71 (d, 2H), 6.49 (s, 1H), 4.07 (m, 2H), 3.16 (s, 1H), 3.00 (s, 6H), 2.41 (s, 3H), 2.26 (m, 2H), 2.05 (m, 1H), 1.73 (m, 5H)

MS (ESI⁺, m/z): 405 [M+H]⁺

### EXAMPLE 8

### Preparation of (E)-6-(2-(4-morpholinoethoxy))-2-(4-dimethylaminostyryl)benzofuran

¹H NMR (CDCl₃, 400 MHz) δ 7.41 (d, 2H), 7.34 (d, 1H), 7.16 (d, 1H), 7.01 (d, 1H), 6.83 (dd, 1H), 6.76 (d, 1H), 6.71 (d, 2H), 6.49 (s, 114), 4.16 (t, 2H), 3.75 (t, 4H), 3.00 (s, 6H), 2.84 (t, 2H), 2.60 (t, 4H)

MS (ESI⁺, m/z): 393 [M+H]⁺

### EXAMPLE 9

### Preparation of (E)-6-(3-((2S,6R)-2,6-dimethylmorpholino)propoxy)-2-(4-dimethylaminostyryl)benzofuran

¹H NMR (CDCl₃, 400 MHz) δ 7.41 (d, 2H), 7.34 (d, 1H), 7.16 (d, 1H), 7.01 (s, 1H), 6.81 (dd, 1H), 6.76 (d, 1H), 6.71 (d, 2H), 6.48 (s, 1H), 4.07 (t, 2H), 3.70 (m, 2H), 3.00 (s, 6H), 2.78 (d, 2H), 2.52 (t, 2H), 2.01 (m, 2H), 1.75 (t, 2H), 1.16 (d, 6H)

MS (ESI⁺, m/z): 435 [M+H]⁺

### EXAMPLE 10

### Preparation of (E)-6-(2-(1-piperidinyl)ethoxy)-2-(4-dimethylaminostyryl)benzofuran

¹H NMR (CDCl₃, 400 MHz) δ 7.41 (d, 2H), 7.34 (d, 1H), 7.16 (d, 1H), 7.01 (d, 1H), 6.82 (dd, 1H), 6.76 (d, 1H), 6.71 (d, 2H), 6.48 (s, 1H), 4.15 (t, 2H), 2.99 (s, 6H), 2.81 (t, 2H), 2.53 (br, 4H), 1.62 (m, 4H), 1.46 (m, 2H)

MS (ESI⁺, m/z): 391 [M+H]⁺

EXAMPLE 11

### Preparation of (E)-6-(3-(1-piperidinyl)propoxy)-2-(4-dimethylaminostyryl)benzofuran

MS (ESI⁺, m/z): 405 [M+H]⁺

### EXAMPLE 12

### Preparation of (E)-6-(1-methyl-2-piperidinylmethoxy)-2-(4-dimethylaminostyryl)benzofuran

¹H NMR (CDCl₃, 400 MHz) δ 7.41 (d, 2H), 7.34 (d, 1H), 7.16 (d, 1H), 7.00 (s, 1H), 6.85 (dd, 1H), 6.76 (d, 1H), 6.71 (d, 2H), 6.48 (s, 1H), 4.06 (m, 2H), 3.00 (s, 6H), 2.94 (m, 1H), 2.41 (s, 3H), 2.33 (s, 1H), 2.18 (m, 1H), 1.83 (m, 2H), 1.62 (m, 3H), 1.33 (m, 1H)

MS (ESI⁺, m/z): 391 [M+H]⁺

### EXAMPLE 13

### Preparation of (E)-6-(1-methyl-3-piperidinylmethoxy)-2-(4-dimethylaminostyryl)benzofuran

¹H NMR (CDCl₃, 400 MHz) δ 7.41 (d, 2H), 7.34 (d, 1H), 7.16 (d, 1H), 6.98 (s, 1H), 6.81 (dd, 1H), 6.76 (d, 1H), 6.71 (d, 2H), 6.48 (s, 1H), 3.87 (m, 2H), 2.95 (m, 8H), 2.34 (s, 3H), 2.24 (br, 1H), 1.90 (m, 5H), 1.16 (m, 1H)

MS (ESI⁺, m/z): 391 [M+H]⁺

### EXAMPLE 14

### Preparation of (E)-6-(3-(4-methyl-1-piperazinyl)propoxy)-2-(4-dimethylaminostyryl)benzofuran

¹H NMR (CDCl₃, 400 MHz) δ 7.41 (d, 2H), 7.34 (d, 1H), 7.16 (d, 1H), 6.98 (s, 1H), 6.81 (dd, 1H), 6.76 (d, 1H), 6.71 (d, 2H), 6.48 (s, 1H), 3.87 (m, 2H), 2.95 (m, 8H), 2.34 (s, 3H), 2.24 (br, 1H), 1.90 (m, 5H), 1.16 (m, 1H)

MS (ESI⁺, m/z): 420 [M+H]⁺

### EXAMPLE 15

### Preparation of (E)-6-(3-(4-thiomorpholinopropoxy))-2-(4-dimethylaminostyryl)benzofuran

¹H NMR (CDCl₃, 400 MHz) δ 7.41 (d, 2H), 7.34 (d, 1H), 7.16 (d, 1H), 7.00 (d, 1H), 6.81 (d, 1H), 6.76 (d, 1H), 6.71 (d, 2H), 6.48 (s, 1H), 4.05 (t, 2H), 3.00 (s, 6H), 2.66 (m, 8H), 2.57 (t, 2H), 2.00 (m, 2H)

MS (ESI⁺, m/z): 423 [M+H]⁺

### EXAMPLE 16

### Preparation of (E)-6-(3-(4-thiomorpholine-1,1-dioxide)propoxy)-2-(4-dimethylaminostyryl)benzofuran

¹H NMR (CDCl₃, 400 MHz) δ 7.41 (d, 2H), 7.34 (d, 1H), 7.16 (d, 1H), 6.99 (s, 1H), 6.78 (m, 2H), 6.71 (d, 2H), 6.49 (s, 1H), 4.06 (t, 2H), 3.05 (m, 8H), 3.00 (s, 6H), 2.73 (t, 2H), 1.98 (m, 2H)

MS (ESI⁺, m/z): 455 [M+H]⁺

### EXAMPLE 17

### Preparation of (E)-6-(3-(4-pyridinyl)propoxy)-2-(4-dimethylaminostyryl)benzofuran

MS (ESI⁺, m/z): 399 [M+H]⁺

### EXAMPLE 18

### Preparation of (E)-6-(3-(3-pyridinyl)propoxy)-2-(4-dimethylaminostyryl)benzofuran

¹H NMR (CDCl₃, 400 MHz) δ 8.52 (s, 1H), 8.47 (d, 1H), 7.55 (d, 1H), 7.41 (d, 2H), 7.35 (d, 1H), 7.23 (dd, 1H), 7.16 (d, 1H), 6.98 (s, 1H), 6.82 (dd, 1H), 6.77 (d, 1H), 6.72 (d, 2H), 6.49 (s, 1H), 4.02 (t, 2H), 3.00 (s, 6H), 2.86 (t, 2H), 2.16 (m, 2H)

MS (ESI⁺, m/z): 399 [M+H]⁺

### EXAMPLE 19

### Preparation of (E)-6-(3-(2-pyridinyl)propoxy)-2-(4-dimethylaminostyryl)benzofuran

¹H NMR (CDCl₃, 400 MHz) δ 8.55 (d, 1H), 7.59 (td, 1H), 7.40 (d, 2H), 7.33 (d, 1H), 7.15 (m, 3H), 6.98 (d, 1H), 6.81 (dd, 1H), 6.76 (d, 1H), 6.71 (d, 2H), 6.44 (s, 1H), 4.04 (t, 2H), 3.01 (m, 8H), 2.27 (m, 2H)

MS (ESI⁺, m/z): 399 [M+H]⁺

### EXAMPLE 20

### Preparation of (E)-6-(methoxy)-2-(4,4′-methyl(2-fluoroethyl)aminostyryl)benzofuran

The procedures used in Steps 1 to 3 of Example 1 were repeated, and the following Steps were conducted to obtain the title compounds.

<Step 4> Preparation of methyl 4-(methylamino)benzoate

4-(methylamino)benzoic acid (12.8 g, 84.7 mmol) was dissolved in methanol (85 mL) at room temperature. AcCl (9.0 mL. 127.0 mmol) was slowly added thereto, and the solution was stirred at reflux for 20 hrs. When the reaction was completed, the solvent was removed by distillation under reduced pressure. The resulting precipitate was suspended in water (500 mL), slightly basified with 1N NaOH to yield the pH 8, stirred for 30 minutes, and then the suspension was filtered. The precipitate was washed with water (100 mL), and dried under reduced pressure to obtain the title compound (13.5 g, 96%).

¹H NMR (DMSO-d6, 400 MHz) δ 7.69 (d, 2H), 6.54 (d, 2H), 3.74 (s, 3H), 2.72 (d, 3H)

<Step 5> Preparation of (4-(methylamino)phenyl)methanol

The compound (3.1 g, 18.6 mmol) obtained in Step 4 above was dissolved in THF (37 mL). A solution prepared by dissolving LiAlH₄ in THF (9.3 mL, 18.6 mmol) was slowly added thereto at 0° C. The resulting mixture was stirred for 8 hrs at 0° C., and an aqueous solution of Na₂SO₄ was added to complete the reaction. Subsequently, a saturated aqueous solution of HCl (50 mL) was added thereto, and the resulting mixture was stirred for 1 hr to form two different layers. The aqueous layer was extracted with EtOAc, and the organic layer was dried over Na₂SO₄, distilled under reduced pressure, and purified by column chromatography (EtOAc/Hexane=1/1 (v/v)) to obtain the title compound (1.4 g, 54%).

¹H NMR (CDCl₃, 400 MHz) δ 7.21 (d, 2H), 6.61 (d, 2H), 4.56 (s, 2H), 2.85 (s, 3H)

<Step 6> Preparation of tert-butyl(4-hydroxymethyl)phenyl)(methyl)carbamate

The compound (1.3 g, 9.7 mmol) obtained in Step 5 above was dissolved in water (2 mL) and dioxane (5 mL). Separately, a solution was prepared by dissolving Boc₂O (3.2 g, 14.6 mmol) in dioxane (3 mL), and the solution was slowly added to the reaction solution, followed by stirring for 16 hrs at room temperature. When the reaction is completed, the solvent was removed by distillation under reduced pressure, and the resulting precipitate was extracted with water and EtOAc. The organic layer was dried over anhydrous MgSO₄, filtered, distilled under reduced pressure and purified by column chromatography (EtOAc/Hexane=¼ (v/v)) to obtain the title compound (2.2 g, 99%).

¹H NMR (CDCl₃, 400 MHz) δ 7.27 (d, 2H), 7.17 (d, 2H), 4.61 (d, 2H), 3.20 (s, 1H), 1.83 (t, 1H), 1.40 (s, 9H)

<Step 7> Preparation of (4-methylamino)phenyl)methanol

The compound (2.3 g, 9.5 mmol) obtained in Step 6 above was dissolved in TEA (4 mL). Separately, a solution was prepared by dissolving sulfur trioxide-pyridine complex (4.5 g, 28.5 mmol) in DMSO (27 mL), and the resulting mixture was slowly added to the reaction solution at 0° C., followed by stirring for 3 hrs at room temperature. When the reaction was completed, water (100 mL) and EtOAc (100 mL) were added thereto, stirred for 1 hr, and the resulting two different layers were separated. The organic layer was dried over anhydrous MgSO₄, filtered, distilled under reduced pressure, and purified by column chromatography (EtOAc/Hexane=¼ (v/v)) to obtain the title compound (2.9 g, 89%).

¹H NMR (CDCl₃, 400 MHz) δ 9.96 (s, 1H), 7.84 (d, 2H), 7.45 (d, 2H), 3.33 (s, 3H), 1.49 (s, 9H)

<Step 8> Preparation of (E)-tert-butyl (4-(2-(6-methoxybenzofuran-2-yl)vinyl)phenyl)(methyl)carbamate

The compound (1.0 g, 3.40 mmol) obtained in Step 7 above was dissolved in THF (20 mL). A solution prepared by dissolving sodium hexamethyldisilazide in THF (3.6 mL, 3.6 mmol) was slowly added to the reaction solution at 0° C., followed by stirring for 2 hrs at 0° C. Separately, the compound obtained in Step 3 above (840 mg, 3.6 mmol) was dissolved in THF (14 mL), and the mixture was slowly added to the reaction solution over 30 min using a syringe pump. The resulting mixture was stirred for 4 hrs at room temperature. MeOH was added thereto at 0° C., and the mixture was distilled under reduced pressure. The residue was extracted with EtOAc and water. The organic layer was dried over anhydrous Na₂SO₄, and filtered. The solvent was removed by distillation under reduced pressure, and the residue was purified by column chromatography (EtOAc/Hexane=¼ (v/v)) to obtain the title compound (607 mg, 47%).

¹H NMR (CDCl₃, 400 MHz) δ 7.46 (d, 2H), 7.39 (d, 2H), 7.19 (d, 2H), 7.02 (d, 1H), 6.91 (d, 2H), 6.59 (s, 1H), 3.87 (s, 3H), 3.28 (s, 3H), 1.47 (s, 9H)

<Step 9> Preparation of (E)-4-(2-(6-methoxybenzofuran-2-yl)vinyl)-N-methylaniline

The compound (590 mg, 1.56 mmol) obtained in Step 8 above was dissolved in MC (16 mL), and TFA (8 μL, 15.6 mmol) was slowly added thereto at 0° C., followed by stirring for 24 hrs at room temperature. When the reaction was completed, the solvent and TFA were removed by distillation under reduced pressure. The resulting precipitate was slightly basified with a saturated NaHCO₃ aqueous solution to yield the pH 8, followed by extraction with MC. The organic layer was dried over Na₂SO₄, and filtered. The solvent was removed by distillation under reduced pressure, and the residue was purified by column chromatography (EtOAc/Hexane=¼ (v/v)) to obtain the title compound (392 mg, 90%).

¹H NMR (CDCl₃, 400 MHz) δ 7.37 (m, 3H), 7.17 (d, 2H), 7.02 (d, 1H), 6.83 (dd, 1H), 6.61 (d, 2H), 6.46 (s, 1H), 3.87 (s, 3H), 2.88 (s, 3H)

<Step 10> Preparation of (E)-6-(methoxy)-2-(4,4′-methyl(2-fluoroethyl)aminostyryl)benzofuran

A solution prepared by dissolving 1-fluoro-2-para-toluenesulfonylethane (772 mg, 3.54 mmol) in sulfolane (8 mL) was slowly added to the compound (330 mg, 1.18 mmol) obtained in Step 9 above. DIPEA (0.8 mL, 4.75 mmol) was added thereto, followed by stirring for 5 hrs at 130° C. After the reaction was completed, the reaction solution was diluted with EtOAc (50 mL), and washed with water to remove sulfolane. The organic layer was dried over Na₂SO₄, filtered, distilled under reduced pressure and purified by column chromatography (EtOAc/Hexane=¼ (v/v)) to obtain the title compound (305 mg, 79%).

¹H NMR (CDCl₃, 400 MHz) δ 7.41 (d, 2H), 7.36 (d, 1H), 7.16 (d, 1H), 7.02 (s, 1H), 6.83 (d, 1H), 6.78 (d, 1H), 6.71 (d, 2H), 6.50 (s, 1H), 4.62 (dt, 2H), 3.86 (s, 3H), 3.69 (dt, 2H), 3.06 (s, 3H)

MS (ESI⁺, m/z): 326 [M+H]⁺

### EXAMPLE 21

### Preparation of (E)-6-(2-(3-oxazolidinyl)ethoxy)-2-(4-dimethylaminostyryl)benzofuran hydrochloride

The compound of (E)-6-(2-(3-oxazolidinyl)ethoxy)-2-(4-dimethylaminostyryl)benzofuran (82 g, 0.202 mol) obtained in Step 6 of Example 1 was added to EtOAc (500 mL), and 1 N HCl in dimethyl ether (800 mL) was added thereto. The reaction solution was stirred for 4.5 hrs at room temperature, washed with EtOAc and diethyl ether, and filtered to obtain the title compound (90 g, 100%).

¹H NMR (MeOD, 400 MHz) δ 7.79 (d, 2H), 7.65 (d, J=8.8 Hz, 2H), 7.51 (d, 1H), 7.26 (s, 2H), 7.23 (d, 1H), 7.00 (dd, 1H), 6.84 (s, 1H), 5.29 (br, 1H), 4.72 (br, 1H), 4.48 (t, 0.5H), 4.45 (t, 1.5H), 4.27 (t, 1.5H), 3.95 (t, 0.5H), 3.83 (m, 3H), 3.53 (m, 1H)

MS (ESI⁺, m/z): 379 (M+H)⁺

Hereinafter, the procedures of Example 21 were repeated by employing respective corresponding starting compounds to obtain the respective title compounds of Examples 22 to 38 having the following analytical data.

### EXAMPLE 22

### Preparation of (E)-6-(2-dimethylaminoethoxy)-2-(4-dimethylaminostyryl)benzofuran hydrochloride

¹H NMR (MeOD, 400 MHz) δ 7.79 (d, 2H), 7.65 (d, 2H), 7.51 (d, 1H), 7.26 (s, 2H), 7.23 (d, 1H), 7.00 (dd, 1H), 6.84 (s, 1H), 4.43 (t, J2H), 3.65 (t, 2H), 3.32 (s, 6H), 3.02 (s, 6H)

MS (ESI⁺, m/z): 351 (M+H)⁺

### EXAMPLE 23

### Preparation of (E)-6-(3-dimethylaminopropoxy)-2-(4-dimethylaminostyryl)benzofuran hydrochloride

¹H NMR (MeOD, 400 MHz) δ 7.79 (d, 2H), 7.67 (d, 2H), 7.46 (d, 1H), 7.24 (s, 2H), 7.13 (d, 1H), 6.91 (dd, 1H), 6.82 (d, 1H), 4.18 (t, 2H), 3.40 (t, 2H), 3.32 (s, 6H), 2.96 (s, 6H), 2.27 (m, 2H)

MS (ESI⁺, m/z): 365 (M+H)⁺

### EXAMPLE 24

### Preparation of (E)-6-(3-diethylaminopropoxy)-2-(4-dimethylaminostyryl)benzofuran hydrochloride

¹H NMR (MeOD, 400 MHz) δ 7.79 (d, 2H), 7.65 (d, 2H), 7.47 (d, 1H), 7.14 (d, 1H), 7.25 (s, 2H), 6.91 (dd, 1H), 6.82 (d, 1H), 4.20 (t, 2H), 3.35 (m, 12H), 3.32 (s, 6H), 2.25 (m, 2H), 1.37 (t, 6H)

MS (ESI⁺, m/z): 393 (M+H)⁺

### EXAMPLE 25

### Preparation of (E)-6-(3-(4-morpholinopropoxy))-2-(4-dimethylaminostyryl)benzofuran hydrochloride

¹H NMR (DMSO-d6, 300 MHz) δ 10.87 (brs, 1H), 7.50 (m, 3H), 7.19 (s, 1H), 7.07 (m, 2H), 6.88-6.84 (m, 3H), 6.77 (m, 2H), 4.36 (m, 2H), 3.99-3.95 (m, 2H), 3.83-3.49 (m, 2H), 3.49-3.45 (d, 2H), 3.31-3.26 (m, 2H), 3.16-3.07 (m, 2H), 2.98 (s, 6H), 2.24-2.19 (quin, 2H)

MS (ESI⁺, m/z): 407 [M+H]⁺

### EXAMPLE 26

### Preparation of (E)-6-(2-(1-pyrrolidinyl)propoxy)-2-(4-dimethylaminostyryl)benzofuran hydrochloride

¹H NMR (MeOD, 400 MHz) δ 7.79 (d, 2H), 7.64 (d, 2H), 7.47 (d, 1H), 7.24 (s, 2H), 7.15 (s, 1H), 6.92 (dd, 1H), 6.82 (s, 1H), 4.23 (m, 2H), 3.73 (m, 1H), 3.61 (m, 1H), 3.33 (s, 6H), 3.21 (m, 1H), 3.00 (s, 3H), 2.52 (m, 2H), 2.14 (m, 3H), 1.93 (m, 1H)

MS (ESI⁺, m/z): 391 (M+H)⁺

### EXAMPLE 27

### Preparation of (E)-6-(2-(4-morpholinoethoxy))-2-(4-dimethylaminostyryl)benzofuran hydrochloride

¹H NMR (MeOD, 400 MHz) δ 7.79 (d, 2H), 7.65 (d, 2H), 7.51 (d, 1H), 7.26 (s, 2H), 7.22 (s, 1H), 6.99 (d, 1H), 6.84 (s, 1H), 6.48 (s, 1H), 4.49 (t, 2H), 4.09 (d, 2H), 3.87 (t, 2H), 3.70 (t, 2H), 3.63 (d, 2H), 3.33 (m, 8H)

MS (ESI⁺, m/z): 393 (M+H)⁺

### EXAMPLE 28

### Preparation of (E)-6-(3-((2S,6R)-2,6-dimethylmorpholino)propoxy)-2-(4-dimethylaminostyryl)benzofuran hydrochloride

¹H NMR (MeOD, 400 MHz) δ 7.78 (d, 2H), 7.64 (d, 2H), 7.46 (d, 1H), 7.24 (s, 2H), 7.12 (s, 1H), 6.89 (dd, 1H), 6.82 (s, 1H), 4.20 (d, 2H), 3.93 (m, 2H), 3.57 (d, 2H), 3.41 (t, 2H), 3.32 (s, 6H), 2.75 (t, 2H), 2.32 (m, 2H), 1.26 (d, 6H)

MS (ESI⁺, m/z): 435 (M+H)⁺

### EXAMPLE 29

### Preparation of (E)-6-(2-(1-piperidinypethoxy)-2-(4-dimethylaminostyryl)benzofuran hydrochloride

¹H NMR (MeOD, 400 MHz) δ 7.78 (d, 2H), 7.65 (d, 2H), 7.50 (d, 1H), 7.25 (s, 2H), 7.21 (d, 1H), 6.98 (dd, 1H), 6.84 (s, 1H), 4.46 (t, 2H), 3.64 (m, 4H), 3.31 (s, 6H), 3.12 (m, 2H), 1.91 (m, 5H), 1.56 (m, 1H)

MS (ESI⁺, m/z): 391 (M+H)⁺

### EXAMPLE 30

### Preparation of (E)-6-(3-(1-piperidinyl)propoxy)-2-(4-dimethylaminostyryl)benzofuran hydrochloride

¹H NMR (MeOD, 400 MHz) δ 7.80 (d, 2H), 7.66 (d, 2H), 7.52 (d, 1H), 7.26 (s, 2H), 7.23 (d, 1H), 7.02 (dd, J1H), 6.85 (s, 1H), 4.56 (dd, 1H), 4.21 (dd, 1H), 3.54 (m, 2H), 3.26 (m, 7H), 2.94 (s, 3H), 2.03 (m, 5H), 1.91 (m, 1H)

MS (ESI⁺, m/z): 391 (M+H)⁺

### EXAMPLE 31

### Preparation of (E)-6-(1-methyl-2-piperidinylmethoxy)-2-(4-dimethylaminostyryl)benzofuran hydrochloride

¹H NMR (MeOD, 400 MHz) δ 7.80 (d, 2H), 7.66 (d, 2H), 7.52 (d, 1H), 7.26 (s, 2H), 7.23 (d, 1H), 7.02 (dd, 1H), 6.85 (s, 1H), 4.56 (dd, 1H), 4.21 (dd, 1H), 3.54 (m, 2H), 3.26 (m, 7H), 2.94 (s, 3H), 2.03 (m, 5H), 1.91 (m, 1H)

MS (ESI⁺, m/z): 391 (M+H)⁺

### EXAMPLE 32

### Preparation of (E)-6-(1-methyl-3-piperidinylmethoxy)-2-(4-dimethylaminostyryl)benzofuran hydrochloride

¹H NMR (MeOD, 400 MHz) δ 7.78 (d, 2H), 7.64 (d, 2H), 7.46 (d, 1H), 7.24 (s, 2H), 7.12 (s, 1H), 6.90 (dd, 1H), 6.82 (s, 1H), 4.10 (m, 1H), 3.96 (m, 1H), 3.72 (d, 1H), 3.55 (d, 1H), 3.31 (s, 6H), 2.97 (m, 5H), 2.39 (m, 1H), 1.98 (m, 31-1), 1.50 (m, 1H)

MS (ESI) m/z 391 (M+H)⁺

### EXAMPLE 33

### Preparation of (E)-6-(3-(4-methyl-1-piperazinyl)propoxy)-2-(4-dimethylaminostyryl)benzofuran hydrochloride

¹H NMR (MeOD, 400 MHz) δ 7.77 (d, 2H), 7.62 (d, 2H), 7.44 (d, 1H), 7.22 (s, 2H), 7.12 (d, 1H), 6.89 (dd, 1H), 6.80 (s, 1H), 4.19 (t, 2H), 3.74 (m, 10H), 3.29 (s, 6H), 3.02 (s, 3H), 2.33 (m, 2H)

MS (ESI⁺, m/z): 420 (M+H)⁺

### EXAMPLE 34

### Preparation of (E)-6-(3-(4-thiomorpholinopropoxy))-2-(4-dimethylaminostyryl)benzofuran hydrochloride

¹H NMR (MeOD, 400 MHz) δ 7.79 (d, 2H), 7.64 (d, 2H), 7.47 (d, 1H), 7.25 (s, 2H), 7.13 (d, 1H), 6.90 (dd, 1H), 6.82 (s, 1H), 4.19 (t, 2H), 3.90 (d, 2H), 3.43 (m, 2H), 3.30 (s, 8H), 3.16 (m, 2H), 2.90 (d, 2H), 2.32 (m, 2H)

MS (ESI⁺, m/z): 423 (M+H)⁺

### EXAMPLE 35

### Preparation of (E)-6-(3-(thiomorpholine-1,1-dioxide)propoxy)-2-(4-dimethylaminostyryl)benzofuran hydrochloride

¹H NMR (MeOD, 400 MHz) δ 7.77 (d, 2H), 7.58 (d, 2H), 7.46 (d, 1H), 7.23 (s, 2H), 7.13 (s, 1H), 6.91 (dd, 1H), 6.81 (s, 1H), 4.21 (t, 2H), 3.92 (br, 4H), 3.58 (m, 6H), 3.32 (s, 6H), 2.34 (m, 2H)

MS (ESI⁺, m/z): 455 (M+H)⁺

### EXAMPLE 36

### Preparation of (E)-6-(3-(4-pyridinyl)propoxy)-2-(4-dimethylaminostyryl)benzofuran hydrochloride

MS (ESI⁺, m/z): 399 (M+H)⁺

### EXAMPLE 37

### Preparation of (E)-6-(3-(2-pyridinyl)propoxy)-2-(4-dimethylaminostyryl)benzofuran hydrochloride

¹H NMR (MeOD, 400 MHz) δ 8.74 (d, 1H), 8.56 (td, 1H), 8.06 (d, 1H), 7.96 (t, 1H), 7.76 (d, 2H), 7.59 (d, 2H), 7.41 (d, Hz, 1H), 7.22 (s, 2H), 7.01 (s, 1H), 6.79 (s 1H), 6.70 (dd, 1H), 4.18 (t, 2H), 3.33 (m, 8H), 2.38 (m, 2H)

MS (ESI⁺, m/z): 399 (M+H)⁺

### EXAMPLE 38

### Preparation of (E)-6-(3-(3-pyridinyl)propoxy)-2-(4-dimethylaminostyryl)benzofuran hydrochloride

¹H NMR (MeOD, 400 MHz) δ 8.83 (s, 1H), 8.74 (d, 1H), 8.62 (d, 1H), 8.05 (dd, 1H), 7.77 (d, 2H), 7.60 (d, 2H), 7.43 (d, 1H), 7.22 (s, 2H), 7.07 (s, 1H), 6.82 (dd, 1H), 6.80 (s, 1H), 4.14 (t, 2H), 3.31 (s, 6H), 3.15 (t, 2H), 2.27 (m, 2H)

MS (ESI⁺, m/z): 399 (M+H)⁺

The compounds obtained in Examples 1 to 20 are represented by the following structural formula, as shown in Table 1 below.

TABLE 1

| Example | Nomenclature | Formula |
|---|---|---|
| 1 | (E)-6-(2-(3-oxazolidinyl)ethoxy)-2-(4- dimethylaminostyryl)benzofuran | 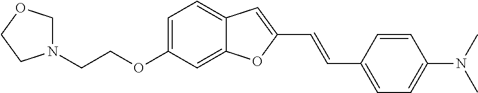 [SMILES: CN(C)c1ccc(/C=C/c2cc3ccc(OCCN4CCOC4)cc3o2)cc1] |
| 2 | (E)-6-(2-dimethylaminoethoxy)-2-(4- dimethylaminostyryl)benzofuran | 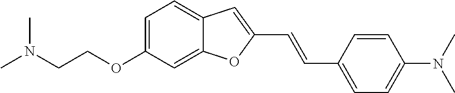 |
| 3 | (E)-6-(3-dimethylaminopropoxy)-2-(4- dimethylaminostyryl)benzofuran | 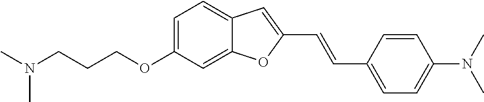 |
| 4 | (E)-6-(3-diethylaminopropoxy)-2-(4- dimethylaminostyryl)benzofuran | 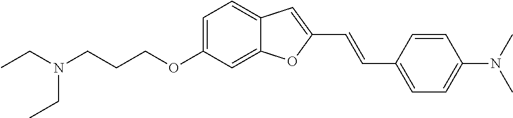 [SMILES: CCN(CC)CCCOc1ccc2cc(/C=C/c3ccc(N(C)C)cc3)oc2c1] |
| 5 | (E)-6-(3-(4-morpholinopropoxy))-2-(4- dimethylaminostyryl)benzofuran | 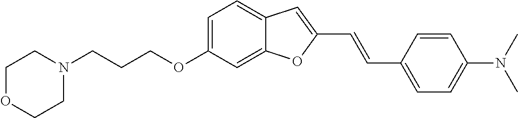 [SMILES: CN(C)c1ccc(/C=C/c2cc3ccc(OCCCN4CCOCC4)cc3o2)cc1] |
| 6 | (E)-6-(2-(1-pyrrolidinyl)propoxy)-2-(4- dimethylaminostyryl)benzofuran | 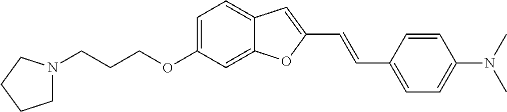 [SMILES: CN(C)c1ccc(/C=C/c2cc3ccc(OCCCN4CCCC4)cc3o2)cc1] |
| 7 | (E)-6-(2-(1-methyl-2-pyrrolidinyl)ethoxy)-2-(4- dimethyl aminostyryl)benzofuran | 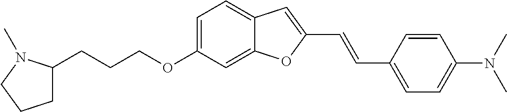 [SMILES: CN(C)c1ccc(/C=C/c2cc3ccc(OCCCC4CCCN4C)cc3o2)cc1] |
| 8 | (E)-6-(2-(4-morpholinoethoxy))-2-(4- dimethylaminostyryl)benzofuran | 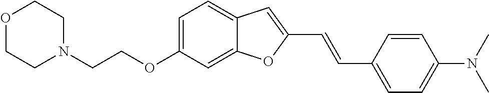 [SMILES: CN(C)c1ccc(/C=C/c2cc3ccc(OCCN4CCOCC4)cc3o2)cc1] |
| 9 | (E)-6-(3-((2S,6R)-2,6- dimethylmorpholino)propoxy)-2-(4- dimethylaminostyryl)benzofuran | 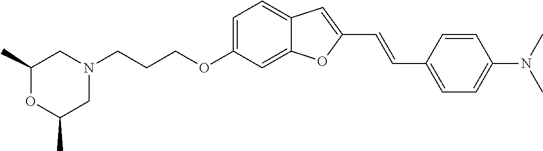 |
| 10 | (E)-6-(2-(1-piperidinyl)ethoxy)-2-(4- dimethylaminostyryl)benzofuran | 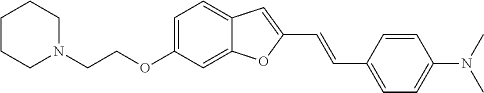 [SMILES: CN(C)c1ccc(/C=C/c2cc3ccc(OCCN4CCCCC4)cc3o2)cc1] |
| 11 | (E)-6-(3-(1-piperidinyl)propoxy)-2-(4- dimethylaminostyryl)benzofuran | 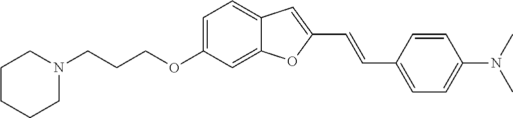 [SMILES: CN(C)c1ccc(/C=C/c2cc3ccc(OCCCN4CCCCC4)cc3o2)cc1] |
| 12 | (E)-6-(1-methyl-2-piperidinylmethoxy)-2-(4- dimethylaminostyryl)benzofuran | 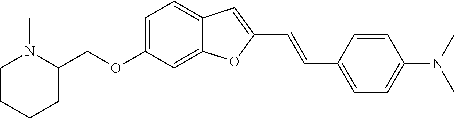 [SMILES: CN(C)c1ccc(/C=C/c2cc3ccc(OCC4CCCCN4C)cc3o2)cc1] |
| 13 | (E)-6-(1-methyl-3-piperidinylmethoxy)-2-(4- dimethylaminostyryl)benzofuran | 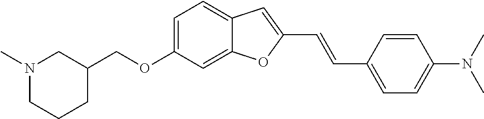 |
| 14 | (E)-6-(3-(4-methyl-1-piperazinyl)propoxy)-2- (4-dimethylaminostyryl)benzofuran | 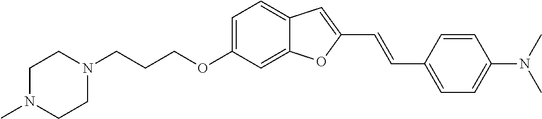 |
| 15 | (E)-6-(3-(4-thiomorpholinopropoxy))-2-(4- dimethylaminostyryl)benzofuran | 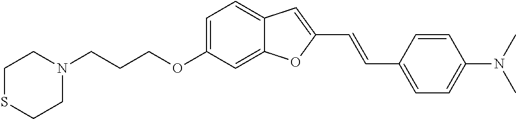 [SMILES: CN(C)c1ccc(/C=C/c2cc3ccc(OCCCN4CCSCC4)cc3o2)cc1] |
| 16 | (E)-6-(3-(4-thiomorpholine-1,1- dioxide)propoxy)-2-(4- dimethylaminostyryl)benzofuran | 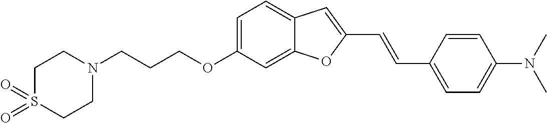 [SMILES: CN(C)c1ccc(/C=C/c2cc3ccc(OCCCN4CCS(=O)(=O)CC4)cc3o2)cc1] |
| 17 | (E)-6-(3-(4-pyridinyl)propoxy)-2-(4- dimethylaminostyryl)benzofuran | 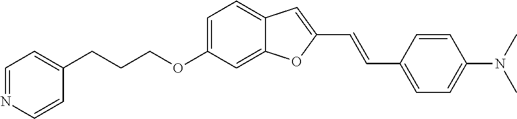 [SMILES: CN(C)c1ccc(/C=C/c2cc3ccc(OCCCc4ccncc4)cc3o2)cc1] |
| 18 | (E)-6-(3-(3-pyridinyl)propoxy)-2-(4- dimethylaminostyryl)benzofuran | 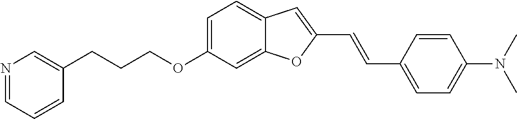 [SMILES: CN(C)c1ccc(/C=C/c2cc3ccc(OCCCc4cccnc4)cc3o2)cc1] |
| 19 | (E)-6-(3-(2-pyridinyl)propoxy)-2-(4- dimethylaminostyryl)benzofuran | 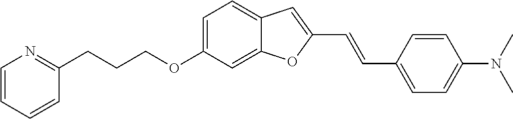 [SMILES: CN(C)c1ccc(/C=C/c2cc3ccc(OCCCc4ccccn4)cc3o2)cc1] |
| 20 | (E)-6-(methoxy)-2-(4,4′-methyl(2- fluoroethyl)aminostyryl)benzofuran | 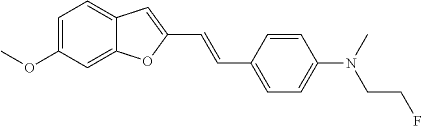 [SMILES: COc1ccc2cc(/C=C/c3ccc(N(C)CCF)cc3)oc2c1] |

The compounds prepared from Examples above were tested for biological assays as follows.

### EXPERIMENTAL EXAMPLE 1

### Inhibitory Effect on the Formation of Beta-Amyloid Fibrils In Vitro (ThT Fluorescence Assay)

In order to investigate the inhibitory effect on the formation of beta-amyloid fibrils, the compounds in accordance with the present invention were examined as follows.

In this experiment, between the two types of beta-amyloid proteins (i.e., beta-amyloid 40 and beta-amyloid 42), beta-amyloid 42 was employed, which is a major target for the development of a therapeutic drug due to its strong neurotoxicity (Hammarstrom, P. et al., Science 2003, 299, 713; and Cai, X. D. et al., Science 1993, 259, 514).

Beta-amyloid 42 (Aβ42) was dissolved in dimethylsulfoxide (DMSO) to form a 250 mM Aβ42 stock solution. In addition, ThT (thioflavin T) was dissolved in distilled water to obtain a concentration of 1 mM and subsequently diluted with 50 mM glycin buffer (pH 8.5) to yield a 5 μM ThT stock solution. 45 μL of PBS (phosphate buffer saline, pH 7.4) was added to each of a 96-well fluorescence microplate (white, F-bottom). 5 μL of the 250 μM Aβ42 stock solution was added to each well. The final concentration of each compound obtained in Examples was in a range of 10 to 0.001 μM by adding 2 μL of a solution, which was prepared by dissolving the subject compounds obtained in Examples in DMSO, to each well. At this time, the final concentration of Aβ42 in each well was 25 μM. The plate was then incubated for 1 hr at room temperature, and 150 μL of the 5 μM ThT stock solution was added to each well.

The fluorescence intensity of each well was determined with the multi-label fluorescence counter (LS-55 Luminescence spectrometer: Perkin Elmer) at an excitation wavelength of 450 nm (excitation slit width: 10 nm) and an emission wavelength of 482 nm (emission slit width: 10 nm), while adjusting counting time to 1 second. The control group was prepared by adding PBS solution, Aβ42 and DMSO, without adding the inventive compound prepared above. A percent inhibition on the formation of beta-amyloid fibrils was calculated in accordance with the following equation, and IC₅₀ was calculated by using GraphPad Prism version 4.03 Program.

% Inhibition=[1−(C−D)/(A−B)]×100

A (control group)=fluorescence intensity in a group treated with PBS solution, Aβ42 and DMSO

B (blank)=fluorescence intensity in a group treated with PBS solution and DMSO

C (experimental group)=fluorescence intensity in a group treated with PBS solution, Aβ42, the inventive compound and DMSO

D (compensation value to the experimental group) =fluorescence intensity in a group treated with PBS solution, the inventive compound and DMSO

% inhibition of the compounds in Examples on the formation of Aβ42 at 10 μM, compared with those of the comparative compounds is shown in Table 2. As a comparative compound, curcumin (Sigma) which is extracted from curry and known to have a potent inhibitory effect against Aβ42 formation was used.

TABLE 2

| | % Inhibition ^{a} |
|---|---|
| Compound | (10 μM) |
| Hydrochloride of Compound 1 (Example 21) | ** |
| Hydrochloride of Compound 2 (Example 22) | *** |
| Hydrochloride of Compound 3 (Example 23) | *** |
| Hydrochloride of Compound 4 (Example 24) | ** |
| Hydrochloride of Compound 5 (Example 25) | *** |
| Compound 6 (Example 6) | * |
| Hydrochloride of Compound 8 (Example 27) | ** |
| Hydrochloride of Compound 9 (Example 28) | * |
| Hydrochloride of Compound 10 (Example 29) | * |
| Compound 11 (Example 11) | * |
| Hydrochloride of Compound 12 (Example 31) | * |
| Hydrochloride of Compound 13 (Example 32) | * |
| Hydrochloride of Compound 14 (Example 33) | * |
| Hydrochloride of Compound 15 (Example 34) | * |
| Hydrochloride of Compound 16 (Example 35) | * |
| Compound 17 (Example 17) | * |
| Hydrochloride of Compound 18 (Example 36) | * |
| Hydrochloride of Compound 19 (Example 37) | * |
| Compound 20 (Example 20) | *** |
| Curcumin | *** |

^{a} % Inhibition: * (<50%),
** (50% ≦ × < 70%),
*** (70%≦)

As shown in Table 2 above, the inventive compounds (e.g., hydrochlorides of Compounds 3 and 5, and Compound 20) showed superior Aβ42% inhibition equal to or greater than that of the comparative compound, curcumin. The remaining compounds also showed inhibitory activity against Aβ42.

### EXPERIMENTAL EXAMPLE 2

### Pharmacokinetics and Passage Test through the Blood-Brain Barrier in Mice and Rat

1. Pharmacokinetics Test

1) Test Animal and Administration of Test Compound

Three 7 week-old ICR mice (weight: approximately 30 g) and three 8 week-old SD rats (weight: approximately 250 g) were used per test group. A solution prepared by dissolving a hydrochloride salt of the compound of Example 3 in excipient (DMSO/Tween 20/saline: 0.1/0.6/2.3, v/v/v) or distilled water was administered to each experimental animal. The test compound was intravenously administered in an amount of 5 mL per kg of body weight through the fine vein or orally administered in an amount of 10 mL per kg of body weight.

2) Blood Concentration Test

At 0.5, 1, 2, 4, 10, and 24 hrs after the oral administration of the test compounds, the blood was collected from periorbital veins into the tube containing heparin (1000 IU/mL, 3 μL) for the mice, and from jugular veins for rats. The blood samples were centrifuged (12,000 rpm for 2 min, Eppendorf Co.) to obtain plasmas and the obtained plasmas were kept in a freezer until the analysis at −80° C.

3) Sample Analysis

The samples were analyzed using LC/MSMS system, and the pretreatment conditions are as follows:

50 μL of plasma was placed in a 2.0 mL tube with a cap (Eppendorf Co.) and acidified by adding 20 μL of 0.1% formic acid thereto. An internal standard solution and 1 mL of ethyl acetate as an extract solvent were added to the resulting solution. The resulting solution was mixed by using a thermomixer (Eppendorf Co.) for 5 min at 1,400 rpm, and then subjected to centrifugation (Eppendorf Co.). The supernatant was collected and concentrated at 35° C. by using a cyclone. The residue was re-dissolved in 50 μL of mobile phase and 5 μL of the resulting solution was injected into LC/MS and analyzed.

2. Passage Test Through the Blood-Brain Barrier

1) Test Animal and Administration of Test Compound

Three 7 week-old ICR mice (weight: approximately 30 g) and three 8 week-old SD rats (weight: approximately 250 g) were used per test group. A solution prepared by dissolving a hydrochloride salt of the compound of Example 3 in excipient (DMSO/Tween 20/saline: 0.1/0.6/2.3, v/v/v) was administered to each experimental animal. The test compound was intravenously administered in an amount of 5 mL per kg of body weight through the fine vein or orally administered in an amount of 10 mL per kg of body weight.

2) Measurement of Concentration in Blood and Tissue (Simultaneous Test)

(1) Blood-Sampling

At 0.5, 1, 2, 4, 10 and 24 hrs after the administration of the test compound, the mice and rats were subjected to insufflations narcosis using isoflorane, followed by cutting the abdomen open. Subsequently, 1 mL of blood was collected from abdominal veins into the tube containing heparin (1000 IU/mL, 3 μL). The obtained blood samples were centrifuged for 2 min at 12,000 rpm to obtain plasma. The obtained plasmas were kept in a freezer at −80° C. until the analysis.

(2) Organ Tissue-Sampling

The mice and rats from which the blood samples were obtained were subjected to bloodletting, and then, the brain tissues of the mice and rats was collected. The brain tissue thus obtained was washed with physiological saline 1 or 2 times to remove blood. The weight of the brain tissue was measured after the removal of adipose tissue and peripheral tissue. 4% bovine serum albumin (BSA) solution diluted with 10-fold was added to the brain tissue. The resulting solution was subjected to homogenization using a homogenizer. The diluted homogenate thus obtained was placed in 2 mL tube and was kept in a freezer at −80° C. until the analysis. All of the treatments to the sample were performed in ice.

(3) Sample Analysis

The samples were analyzed using LC/MSMS system under the following conditions.

50 μL of plasma was placed in a 2.0 mL tube with a cap (Eppendorf Co.). An internal standard solution and 1 mL of ethyl acetate as an extract solvent were added to the resulting solution. The resulting solution was mixed by using thermomixer (Eppendorf Co.) for 5 min at 1400 rpm, and then subjected to centrifugation (Eppendorf Co.). The supernatant was collected and concentrated at 35° C. by using a cyclone. The residue was re-dissolved in 50 μL of mobile phase and 5 μL of the resulting solution was injected into LC/MS and analyzed.

3. Results

The results of pharmacokinetics and passage test through the blood-brain barrier in mice and rats of hydrochloride of the compound of Example 3 are shown in Table 3.

In Table 3, “iv” refers to intravenous injection; “po” refers to per oral; “AUC plasma” refers to an area under the plasma level-time curve; “Cmax” refers to a maximum plasma concentration; “Tmax” refers to a time to reach Cmax; “BA” refers to bioavailability (%) according to Equation 2 below; “AUC Brain” refers to an area under the brain tissue level-time curve; and “AUCBrain/AUCPlasma” refers to a passage rate of the test compound to the brain.

[Formula II]

Bioavailability (%)=[(AUCpo/AUCiv)×(Doseiv/Dosepo)×100]

In the above Equation, AUCpo refers to an area under the blood concentration time curve (AUC) after per oral administration, AUCiv means an AUC after the intravenous injection; Doseiv refers to a dose of the intravenous injection; and Dosepo refers to a dose of the per os.

TABLE 3

| | Hydrochloride of the compound | |
|---|---|---|
| | of Example 3 (n = 3) | |
| Parameter | 5.0 mg/kg, iv | 10.0 mg/kg, po |
| AUC Plasma (ng · h/ml) | 534.7 | 472.1 |
| Cmax(ng/ml) | — | 62.7 |
| Tmax (hr) | — | 4 |
| BA (%) | 43.4 | |
| AUC Brain (ng · h/ml) | 54,114.7 | 27,412 |
| Cmax (ng/ml) | | 3,828 |
| Tmax (hr) | | 10 |
| AUCBrain/AUCPlasma | 101.2 | 58.1 |

As can be seen from Table 3, hydrochloride of the compound of Example 3 in accordance with the present invention showed a high degree of AUC, which is suitable for a therapeutic agent for brain diseases, as well as superior bioavailability.

Also, as shown in the result of passage test through the blood-brain barrier, it is found that the compound of Example 3 demonstrated 100% or more of passage ability compared with plasma, which is suitable for a therapeutic agent for brain diseases.

### EXPERIMENTAL EXAMPLE 3

### Pharmacokinetics Test in Mice (Dose-Dependent)

1) Test Animal and Administration of Test Compound

Two 6 week-old ICR mice (weight: approximately 25 g) were used per test group. A solution prepared by dissolving the compound of Example 3 in distilled water was administered to each experimental animal. The test compound was orally administered in an amount of 10 mL per kg of body weight.

2) Blood Concentration Analysis

At 0.5, 1, 2, 4, 10 and 24 hrs after the oral administration of the test compound, the blood was collected from jugular veins into a tube containing heparin (1,000 IU/mL, 3 μL). The obtained plasmas were centrifuged (12,000 rpm, 2 min, Eppendorf), and contained in a freezer at −80° C. until analysis.

3) Sample Analysis

The samples were analyzed by using LC/MSMS system under the following conditions.

50 μL of plasma was placed in a 2.0 mL tube with a cap (Eppendorf Co.) and acidified by adding 20 μL of 0.1% formic acid thereto. An internal standard solution and 1 mL of ethyl acetate as an extract solvent were added to the resulting solution. The resulting solution was mixed by using thermomixer (Eppendorf Co.) for 5 min at 1400 rpm, and then subjected to centrifugation (Eppendorf Co.). The supernatant was collected and concentrated at 35° C. by using a cyclone. The residue was re-dissolved in 50 μL of mobile phase and 5 μL of the resulting solution was injected into LC/MS and analyzed.

4) Results

The results of pharmacokinetics in mice (dose-dependent) are shown in Table 4.

In Table 4, “po” refers to per oral; “AUC plasma” refers to an area under the plasma level-time curve; “Cmax” refers to a maximum plasma concentration; and “Tmax” refers to a time to reach Cmax.

TABLE 4

| | Compound of Example 3 | | |
|---|---|---|---|
| Parameter | 10 mg/kg, po | 30 mg/kg, po | 100 mg/kg, po |
| AUC Plasma | 442.6 | 1887.5 | 4582.9 |
| (ng · h/ml) | | | |
| Cmax (ng/ml) | 40.4 | 188.5 | 378.5 |
| Tmax (hr) | 0.5 | 2.0 | 4.0 |
| Half-life (hr) | 4.9 | 5.4 | 23.5 |
| AUC ratio¹ | 1 | 4.2 | 10.4 |
| Cmax ratio² | 1 | 4.7 | 9.4 |

Mean ± SD, * NC not calculated,
¹AUC result (30 or 100 mg/kg)/AUC result (10 mg/kg),
²Cmax result (30 or 100 mg/kg)/Cmax result (10 mg/kg), ( ): Dose ratio

Styrylbenzofuran compounds as disclosed in Korean Patent Laid-open Publication No. 2009-0129377 did not show linear dose-dependent pharmacokinetics. In contrast, the compound of Example 3, as shown in Table 4, improved AUC_{0-24 hr} after oral administration of 10 mg/kg, 30 mg/kg, and 100 mg/kg in mice, from 1.0:3.0:10.0-fold to 1.0:4.2:10.4-fold, and hence showed linear pharmacokinetics.

### EXPERIMENTAL EXAMPLE 4

### Inhibitory Effect of hERG Potassium Ion Channel

1) Model Cell Line and Culture

A HEK-hERG cell line (IonGate Biosciences, Frankfrut, Germany) which stably expresses hERG was cultured in a DMEM (Dulbecco's modified Eagle's Medium, Sigma Co., St. Louis, Mo., USA) supplemented with 10% fetal bovine serum (FBS, Cambrex, Walkesville, Md., USA) and 0.5 mg/mL zeocin (Invitrogen, Carlsbad, Calif., USA). The cell line was subcultured for 5 days after culture when 80% confluency was reached.

2) Preparation of Test Solution and Test Drug

(1) Test Solution

A solution within an electrode used to measure the potassium ion current is composed of 115 mM K-aspartate, 20 mM KCl, 10 mM EGTA, 10 mM HEPES, 2.5 mM tris-phosphocreatine, 0.1 mM Na₂GTP and 5 mM MgCl₂ (pH 7.2, 290 mOsm/Kg H₂O). A solution for an extracellular perfusate is composed of 135 mM NaCl, 5 mM KCl, 1 mM MgCl₂, 2 mM CaCl₂, 10 mM glucose and 10 mM HEPES (pH 7.2, 300 mOsm/Kg H₂O).

(2) Test Drug

Test drug solutions were prepared by respectively diluting the inventive compounds with extracellular perfusate to a desired concentration. The prepared test drug solution was placed in a 7-array polyethylene tube which is connected into a capillary column for gas chromatography and was dropped from the tip of the column at a height of 100 μm or less to the HEK-hERG cell line.

3) Ion Current Measurement

Potassium ion current was measured by using EPC10 (Instrutech Co., NY, USA) patch clamp amplifier in accordance with the conventional whole-cell patch clamp method. An electrode used in the measurement was a borosilicate glass capillary (external diameter: 1.65 mm, inside diameter: 1.2 mm, Corning 7052, Gamer Glass Co., Claremont, Calif., USA) prepared by using a P-97 Flaming-Brown micropipette puller (Sutter Instrument Co.). The electrode was coated with Sylgard 184 (Dow Corning Co., Midland, Mich., USA) and trimmed with microforge (Narishige Co., Tokyo, Japan). The electrode had a resistance of 2 to 3 MΩ when filled with a solution. A culture dish containing HEK-hERG cells was placed in an inverted microscope (Nikon Co.) and extracellular perfusate containing the inventive compound was perfused at a rate of 1 to 2 mL/min. The membrane capacitance and series resistance of cell membrane were calibrated by 80% or more and potassium ion current was measured at a sampling rate of 2 kHz and a low-pass filter of 2 kHz (−3 dB; 8-pole Bassel filter). The test was conducted at room temperature (21 to 24° C.).

4) Data Analysis and Statistics

The results were analyzed by using Pulse/Pulsefit (v9.0, HEKA Elektronik, Lambrecht, Germany) and Igor macro. The results were given as mean±standard error. IC₅₀ of a test compound, the concentration of the test compound at which 50% ion current was inhibited, was obtained from a concentration-response curve by using the Hill equation [Block=(1+IC₅₀/[drug]ⁿ])⁻¹]. The result is found in Table 5 below.

TABLE 5

| | Inhibitory activity |
|---|---|
| Compound | (IC₅₀) |
| Hydrochloride of Compound 2 | 52.14 ± 3.68 μM |

As shown in Table 5, the inventive compound of Example 2 showed an insignificant inhibitory efficacy on hERG potassium ion channel, and thus they are considered to be non-cardiotoxic.

### EXPERIMENTAL EXAMPLE 5

### Solubility Measurement by Using HPLC (Liquid Chromatography)

1) Preparation of Saturated Solution

Saturated solutions were prepared by dissolving each of the compounds of Examples 3 to 5 and the styrylbenzofuran compound of KR Pat. Laid-open Publication No. 2009-0129377 (Example 9) in water (10 mL). The samples were stirred for 30 minutes at 99 rpm in F6 mode by using a mixing system (MYLAB Rotamix, SLPM-2M). In the case where a solute is completely dissolved in a solvent after the stirring, a saturated solution with high concentration for solubility measurement was prepared again to prevent the solution from becoming transparent even after the stirring process.

2) Pretreatment of Sample

Saturated solutions (5 mL or more) were centrifuged for 5 minutes at 4,000 rpm by using a centrifugal separator. The filtration process was conducted by using a 0.2 μm PTFE membrane filter.

3) Analysis Using Liquid Chromatography

Standard solutions of drugs being tested are prepared according to quantitative analysis, and test samples were then diluted with an acetonitrile solvent, followed by liquid chromatography analysis.

4) Results

The styrylbenzofuran compounds in accordance with Korean Patent Laid-open Publication No. 2009-0129377 exhibited a solubility of 0.00005 mg/mL or less in water. However, the inventive styrylbenzofuran compounds of Examples 3 to 5 showed a solubility of 1 mg/mL or greater in water.

As can be seen in the results of Experimental Examples above, styrylbenzofuran compounds in accordance with the present invention have inhibitory activity against beta-amyloid fibril formation, and thus can be used singly or in combination with other drugs for the prevention and treatment of degenerative brain diseases including senile dementia. The inventive compounds have improved solubility as compared to the conventional styrylbenzofuran compound (Korean Patent Laid-open Publication No. 2009-0129377), thereby displaying linear kinetics, and also have an advantage of minimizing the adverse side effects.

What is claimed is:

1. A compound selected from the group consisting of an aminostyrylbenzofuran compound of Formula (I) below, a pharmaceutically acceptable salt, an isomer, a hydrate, and a solvate thereof: 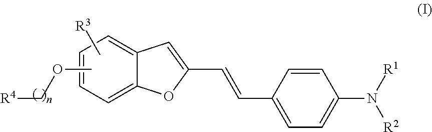
wherein,
R¹ and R² are each independently hydrogen, C₁₋₆ alkyl, or C₃₋₈ cycloalkyl, wherein the C₁₋₆ alkyl or C₃₋₈ cycloalkyl is optionally substituted with one or more halogens;
R³ is hydrogen, C₁₋₃ alkyl or C₁₋₃ alkoxy;
R⁴ is hydrogen, NR⁵R⁶, C₃₋₈ heterocycloalkyl comprising 1 or more N, and randomly comprising O and S, or pyridyl, wherein the C₃₋₈ heterocycloalkyl or pyridyl is optionally substituted with one or more substituents selected from the group consisting of C₁₋₃ alkyl and oxo, and said R⁵ and R⁶ are each independently hydrogen or C₁₋₆ alkyl; and
n is an integer ranging from 1 to 4.

2. The compound of claim 1, wherein R¹ is methyl, and R² is methyl or 2-fluoro ethyl.

3. The compound of claim 1, wherein R³ is hydrogen or C₁₋₂ alkoxy.

4. The compound of claim 1, wherein R⁴ is hydrogen, dimethylamine, diethylamine, pyridin-2-yl, pyridin-3-yl, pyridin-4-yl, piperidin-1-yl, 1-methyl piperidin-2-yl, 1-methyl piperidin-3-yl, oxazolidin-3-yl, pyrrolidin-1-yl, 1-methyl pyrrolidin-2-yl, morpholin-4-yl, (2S,6R)-2,6-dimethylmorpholin-4-yl, 1 -methylpiperazin-4-yl, thiomorpholin-4-yl, or 1,1-dioxothiomorpholin-4-yl.

5. The compound of claim 1, wherein n is an integer ranging from 1 to 3.

6. The compound of claim 1, wherein the aminosstyrylbenzofuran compound of Formula (I) is selected from the group consisting of:
1) (E)-6-(2-(3-oxazolidinyl)ethoxy)-2-(4-dimethylaminostyryl)benzofuran;
2) (E)-6-(2-dimethylaminoethoxy)-2-(4-dimethylaminostyryl)benzofuran;
3) (E)-6-(3-dimethylaminopropoxy)-2-(4-dimethylaminostyryl)benzofuran;
4) (E)-6-(3-diethylaminopropoxy)-2-(4-dimethylaminostyryl)benzofuran;
5) (E)-6-(3-(4-morpholinopropoxy))-2-(4-dimethylaminostyryl)benzofuran;
6) (E)-6-(2-(1-pyrrolidinyl)propoxy)-2-(4-dimethylaminostyryl)benzofuran;
7) (E)-6-(2-(1-methyl-2-pyrrolidinyl)ethoxy)-2-(4-dimethylaminostyryl)benzofuran;
8) (E)-6-(2-(4-morpholinoethoxy))-2-(4-dimethylaminostyryl)benzofuran;
9) (E)-6-(3-((2S,6R)-2,6-dimethylmorpholino)propoxy)-2-(4-dimethylaminostyryebenzofuran;
10) (E)-6-(2-(1-piperidinyl)ethoxy)-2-(4-dimethylaminostyryl)benzofuran;
11) (E)-6-(3 -(1- piperidinyl)propoxy)-2-(4-dimethylaminostyryl)benzofuran;
12) (E)-6-(1-methyl-2-piperidinylmethoxy)-2-(4-dimethylaminostyryl)benzofuran;
13) (E)-6-(1-methyl-3-piperidinylmethoxy)-2-(4-dimethylaminostyryl)benzofuran;
14) (E)-6-(3-(4-methyl-l-piperazinyl)propoxy)-2-(4-dimethylaminostyrypbenzofuran;
15) (E)-6-(3-(4-thiomorpholinopropoxy))-2-(4-dimethylaminostyryl)benzofuran;
16) (E)-6-(3-(4-thiomorpholine-1,1-dioxide)propoxy)-2-(4-dimethylaminostyryl)benzofuran;
17) (E)-6-(3-(4-pyridinyl)propoxy)-2-(4-dimethylaminostyryl)benzofuran;
18) (E)-6-(3-(3-pyridinyl)propoxy)-2-(4-dimethylaminostyryl)benzofuran;
19) (E)-6-(3-(2-pyridinyl)propoxy)-2-(4-dimethylaminostyryl)benzofuran; and
20) (E)-6-(methoxy)-2-(4,4′-methyl(2-fluoroethyl)aminostyryl)benzofuran.

7. The compound of claim 1, wherein the pharmaceutically acceptable salt is acid addition salts formed by an acid selected from the group consisting of hydrochloric acid, hydrobromic acid, sulfuric acid, phosphoric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid, p-toluenesulfonic acid, hydriodic acid, formic acid, trichloroacetic acid, trifluoroacetic acid, gluconic acid and naphthalenesulfonic acid

8. A pharmaceutical composition for the inhibition of beta-amyloid fibril formation comprising the compound of claim 1 as an active ingredient.

9. A pharmaceutical composition for the prevention or treatment of degenerative brain diseases which comprises the compound of claim 1 as an active ingredient.

10. The pharmaceutical composition of claim 9, wherein the degenerative brain disease is senile dementia, stroke, Parkinson's disease, Huntington's disease, or mad cow disease.

11. A use of the compound of claim 1 for the manufacture of a medicament for preventing or treating degenerative brain diseases.

12. A method for preventing or treating degenerative brain diseases, which method comprises administering the compound of claim 1 to a mammal in need thereof.
